(19) United States
(12) Patent Application Publication
(10) Pub. No.: US 2013/0204103 A1
(43) Pub. Date: Aug. 8, 2013

(54) MEDICAL DEVICE SYSTEM

(71) Applicant: Albert Maarek, Miami, FL (US)

(72) Inventor: Albert Maarek, Miami, FL (US)

(21) Appl. No.: 13/514,353

(22) PCT Filed: Dec. 6, 2010
(86) PCT No.: PCT/IB10/03114

§ 371 Date: Oct. 10, 2012

Related U.S. Application Data
(60) Provisional application No. 61/267,510, filed on Dec. 8, 2009.
(60) Provisional application No. 61/267,542, filed on Dec. 8, 2009.

(51) Int. Cl. A61B 5/1455; A61B 5/0245; A61B 5/091; A61B 5/145; A61B 5/053

(52) U.S. Cl. CPC A61B 5/14551; A61B 5/14532; A61B 5/053; A61B 5/091; A61B 5/0245
USPC 600/323; 600/476; 600/365

(57) ABSTRACT

The invention provides a medical device system comprising at least two technologies wherein at least one technology is based on bio-impedance measuring and/or at least one technology is based on spectrophotometry measurements wherein software cross analyses the results to assess the homeostasis of an individual. The technologies measure a variety of parameters. In one embodiment the bioimpedance measuring equipment measures in bipolar mode and in tetrapolar mode and the spectophotmeter measuring equipment comprises a pulse oximeter. The system and homeostasis score can be used to determine and monitor therapy for a patient.

The present invention relates to a medical device system utilising a combination of technologies and software to establish an evaluation. More particularly the device comprises technologies including spectrophotometry and impedance monitoring to establish a measure of homeostasis for a practitioner to determine and monitor treatment.

As stated by Lippincott (Medical encyclopedia): “Disease or death is often the result of dysfunction of internal environment and regulatory mechanisms. Understanding the body's processes, responses and functions is clearly fundamental to the intelligent practice of medicine.” At present, the clinical context, the lab tests, functional tests such as EKG or Doppler and imagery provide doctors data to establish diagnoses and treatment plans on predictions based upon statistical averages.

However, these averages do not take into account the overall condition of any individual patient. An overall homeostasis evaluation which represent a patient's potential adaptation to a dysfunction or disease should enhance a treatment plan.

It is an aim of the present invention to provide a device or series of devices comprising different technologies to establish an overall condition of the patient. It is a further aim to assign a score to be known as the homeostasis score.

The homeostasis score provides a fast overview of a patient's homeostasis processes and responses with the key indicators, to understand the patient's potential adaptation to lifestyle, disorders, diseases or current treatment.

The healthy subject is not identified as such simply because he does not have any disease, but because his homeostasis score is acceptable and therefore his body can adapt and remain healthy when challenged. The homeostasis score cannot be used as diagnosis.

The proposed technology and its analysis aims to provide low cost therapeutic follow up. Therefore, with the adjunct of the homeostasis evaluation, a doctor should be able to test how the planned treatment would affect a patient, save time and as the possibilities of treating diseases improve, it is important to choose the right treatment for each individual patient.

According to the present invention there is provided a device wherein at least one technology is based on bio-impedance measuring and/or at least one technology is based on spectrophotometry measurements and software cross analyses the results to assess the homeostasis of an individual.

Medical device monitoring systems tend to measure one parameter or set of parameters in isolation. This has disadvantages for the patient in that other conditions or aggravating issues could be overlooked.

The present invention provides a medical device and or a series of medical devices measuring a variety of parameters using different technologies and software to provide a homeostasis score.

According to the present invention there is provided a medical device comprising at least a pulse oximeter which provides a vascular waveform. in combination with other biosensors and software.

The devices combined in one or more devices comprising a system (to calculate the homeostasis score) may include EKG, blood glucose meter, spirometer and a variety of other known and new technologies.

In one particular preferred embodiment the system is a combination of 4 biosensor technologies with 6 features and signal processing analysis managed by software.

Preferred technologies include a) bioimpedance in bipolar mode (EIS sensor), b) bioimpedance in tetrapolar mode (ES-BC sensor), c) the spectrophotometry (ESO sensor) and d) oscillometric measurements. (NIBP sensor)

### Preferred Bio Impedance Biosensor Features:

The bio impedance in bipolar mode sensor (such as the EIS (electro interstitial scan) sensor) feature evaluates the segmental and general conductivity of the human body with low frequencies via at least 4 to 8 tactile electrodes. The signal processing analysis of the measurement provides estimated parameters related to living tissue: interstitial fluid sodium ion related to the Na+/K+ATPase pump activity (NAKA), interstitial fluid negative ions (chloride ions and bicarbonate) and morphology of the interstitial fluid space.

The bio impedance in tetra polar mode (ES-BC (electro scan body composition) sensor) feature evaluates the resistance and the reactance of the human body using a mono frequency (50 KHz) via 4 tactile electrodes, to estimate body composition parameters (total body water (TWB), fat free mass, fat mass) according to predictive equations as commonly seen in peer reviews. (W C Chumlea, S S Guo, R J Kuczmarski, K M Flegal, C L Johnson, S B Heymsfield, H C Lukaski, K Friedl and V S Hubbard Body composition estimates from NHANES III bioelectrical impedance data. International Journal of Obesity (2002) 26, 1596-1609)

### Preferred Spectrophotometry Measurement Features:

The pulse oximeter (ESO sensor) displays SpO2%, pulse rate value and vertical bar graph pulse amplitude.

The photoelectrical plethysmograph or digital pulse analysis (DPA) feature is the signal processing analysis of the pulse waveform provided by the oximeter. The mathematical analyses provide indicators to estimate the artery stiffness, associated with the heart rate detection the cardiac output and associated with the NIBP sensor, the systemic vascular resistance and means arterial pressure.

The Heart Rate Variability feature (HRV), both in the time domain and in the frequency domain (spectral analysis). Each QRS complex is detected and the so-called normal-to-normal (NN) or Rate-to-Rate (RR) intervals between adjacent QRS complexes are the result of sinus node depolarization. The signal processing analysis of the measurement provides indicators to estimate the ANS (Autonomic Nervous System) activity.

### Preferred Oscillometric Measurements.

The non invasive blood pressure device (NIBP sensor) feature is the measurement of the systolic and diastolic pressure.

The invention will now be described with reference to the accompany non-limiting figures wherein

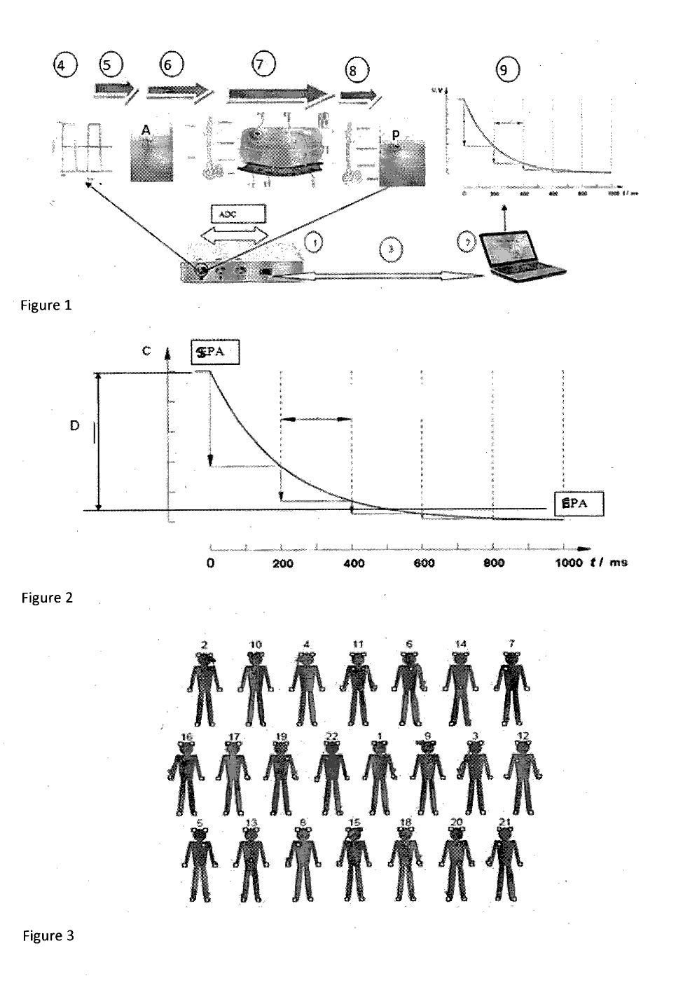

FIG. 1 shows the EIS process

FIG. 2 shows a graph of conductivity against time for an individual EIS measurement

FIG. 3 shows the pathways of the individual EIS measurements

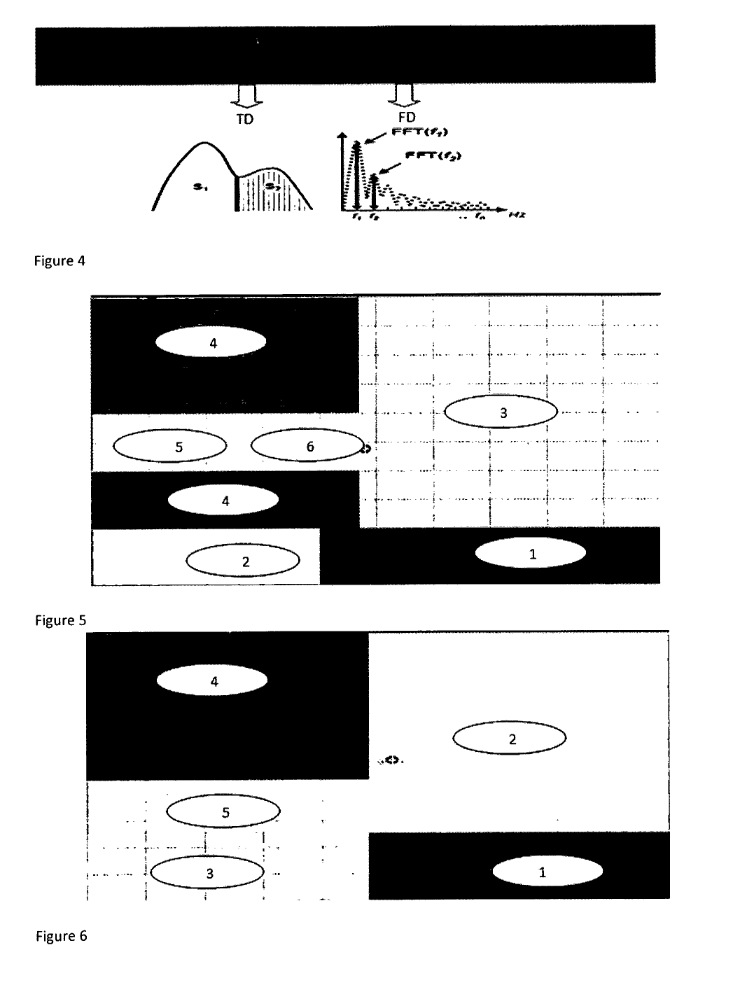

FIG. 4 shows the HRV signal and time domain and frequency domain analysis

FIG. 5 shows the body system tissue diagram with zones marked to assess risk

FIG. 6 shows the brain system tissue diagram with zones marked to assess risk

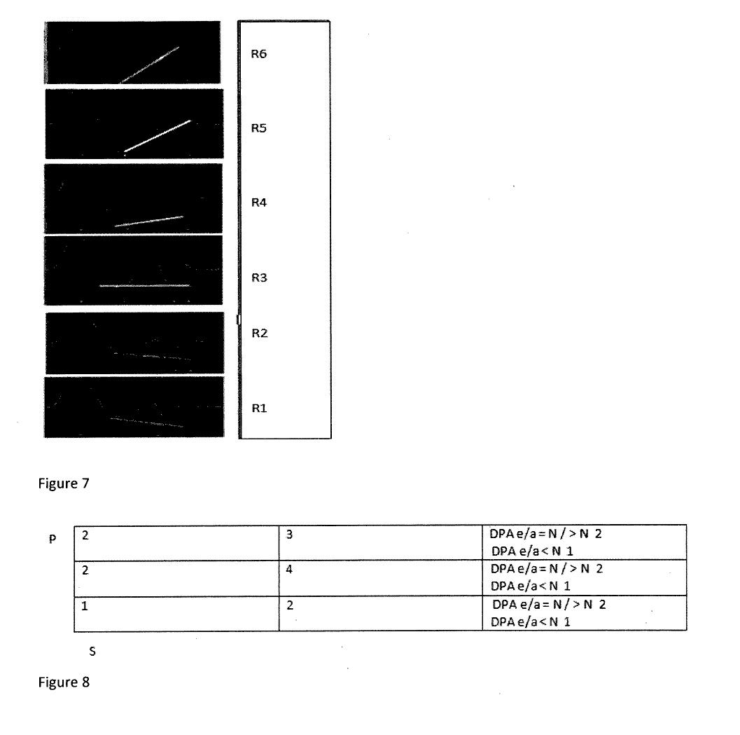

FIG. 7 shows the photoelectrical plesthysmography or DPA class risk

FIG. 8 shows class risk from HRV assessment

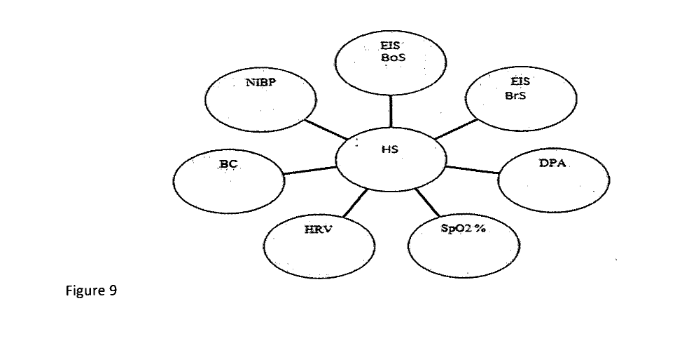

FIG. 9 shows the various elements contributing to the calculation of the homeostasis score

### BIO IMPEDANCE BIPOLAR MODE (EIS SENSOR) TECHNOLOGY

### General Principles

EIS sensor is a programmable electro medical system (PEMS) including:
USB plug and play hardware devices including interface box, disposable electrodes, reusable plates and reusable cables
Software installed on a computer.

Successive measurements are typically made with weak Direct Current and very low frequency (700 Hz) between six tactile electrodes placed symmetrically on the left and right forehead, palm of hands, and sole of the feet of the subject.

The hand and foot electrodes are typically at least 250 cm2 and in metal

The forehead electrodes are typically disposable (single use) and preferably in AgAgCl.

Each electrode is alternatively cathode then anode (bipolar mode), which permits in the particular embodiment described the recording of the intensity/voltage/resistance and conductivity (Law of Ohm) of 11 segments (segments means interstitial fluid pathways) of the human body.

In this case odd numbered segments are measured from the anode to the cathode and even segments are measured from the cathode to anode.

### Features and Intended Uses

### According to the Features

The measurements relate to estimations of parameters related to living tissue:
Estimation of the interstitial fluid sodium ions density related to the Na+/K+ATPase pump activity (NAKA),
Estimation of the interstitial fluid negative ions density (chloride ions and bicarbonate)
Estimation of the morphology of the interstitial fluid space.

### According to the Clinical Investigations:

Follow ups of drugs' administrations (thyroid hormone, beta blockers, ACE inhibitors and SSRI treatments)
Adjunct in diagnosis of ADHD children with the conventional methods
Adjunct to PSA test and DRI prostate analysis of men
Estimation of the sympathetic system modulation

The EIS may be used for children (over 5 years) and adult patients.

The device is not intended for use in life support situations and is not for continuous monitoring. The system should be used by a practitioner taking into account the clinical context of each individual patient.

Data Acquisition Diagram: Description for One Segment from Anode (Active Electrode) to Cathode (Passive Electrode) FIG. 1.

### FIG. 1 Description:

1. Hardware
2. Software installed in PC
3. Communication Protocol via USB
   Sending of the output signal waveform to the active electrode (AE). The signal waveform is rectangular, is continuous during 1 or 3 seconds/per human body segment located between 2 electrodes. Each electrode is alternatively anode then cathode for each segment/pathway
   This operation is realized 22 times (11 pathways) according to a programmed sequence. Current specification: DC and Frequency 700 Hz, voltage U (output)= or >1.2 V and I (intensity)= or >12 μA. Time between each pulse (resolution)=<30 ms
4. Entrance of the current through the skin via the eccrine sweat gland
5. Pathway of the current into the body located between the 2 electrodes: Interstitial fluid
6. Exit of the current through the skin via the eccrine sweat gland
7. Current transmitted to the passive electrode (PE) and transfers at the measured current to the hardware=>ADC cheap=>USB port=>Software
8. The software receives 32 or 255 measurements according to the time of current application, converts the intensity and voltage into conductivity according to Ohm's law and generates a graph for each sequence of measurement.
   Analysis of the graph of conductivity generated by the software for each sequence of measurement. FIG. 2

### FIG. 2 Description:

EPA=First value of measured conductivity for each segment
SPA=Last value of conductivity for each segment
The delta EPA-SPA=Dispersion of the current
The selected conductivity value for each sequence of measurement is the value SPA (After stabilization of the measurement)
The curve can be straight or inverse. This curve is similar to the chronoamperometry measurement which is an electrochemical measurement (intensity related with a chemical substance concentration i.e. below)

Sequence of measurement and pathways between the left and right forehead, hands and feet segments in this embodiment are as shown in FIG. 3

### FIG. 3 Description:

The current is sent from the anode to cathode for the odd numbered segments 1/3/5/7/9/11/13/15/17/19/21

The current is sent from the cathode to anode for the even segments 2/4/6/8/10/12/14/16/18/20/22

This sequence is a programmed sequence and can be changed and this change does not affect the results of the device.

### Signal Processing Analysis

1. Domain Analysis: Results analysis for each segment/pathway
a. The full cycle comprises the measurement of the 11 segments/pathways measured in the polarity anode-cathode and in second time in the polarity cathode-anode. This operation is performed 4 times (m1, m2, m3 and m4) 2 measurements in DC and 2 measurements with a very low frequency 700 Hz. The graph is an average of the 4 measurements.
b. SDC+=Conductivity in μS of each odd numbered segment/pathway normal range 8 to 18 μS and pathway brain (segment 9/10) normal range 3.40 to 10.33 μS
c. SDC−=Conductivity in μS of each even segment/pathway normal range 8 to 18 μS and pathway brain (segment 9/10) normal range 3.40 to 10.33 μS
d. EPA-SPA α parameter=Dispersion in C.U of each segment/pathway pathway body normal range 0.60 to 0.67 and pathway brain normal range 0.65 to 0.70. (Calculation from the Cole-Cole equation)
2. Frequency or spectral analysis: Results
a. The full cycle comprises the measurement of the 11 segments/pathways measured in the polarity anode-cathode and in second time in the polarity cathode-anode. This operation is performed 4 times (m1, m2, m3 and m4) 2 measurements in DC and 2 measurements with a very low frequency 700 Hz. The graph is an average of the 4 measurements. The conductivity measurements are in abscissa and segments in ordinate.
b. Application of the Fast Fourier Transform (FFT) to the entire signal
c. Components of the FFT: EIS HF, EIS LF, EIS VLF.
   EIS HF (High frequencies from 0.1875 to 0.50 Hz). Normal range from 22 to 34%.
   EIS LF (Low frequencies from 0.05 to 0.1875 Hz). Normal range from 22 to 46%. EIS
   VLF (Very Low frequencies from 0 to 0.05 Hz). Normal range from 22 to 50%.
   EIS HF/VLF ratio. Normal range from 0.44 to 1.54.

### EIS Features

a. The entrance and exit of the EIS current are the eccrine sweat glands and the system operates via the large tactile planar electrodes in the parts the body with the higher density of sweat glands (palms of hands, soles of feet and left and right forehead).
b. The EIS Technology uses a very low frequency close to the DC, therefore, the current flows around the cells very close to the cell membrane in the area of the interstitial fluid and does not penetrate the cell in accordance with the fickle circuit and peer reviews related to the BIA (Bio Impedance Analysis). This fact is confirmed by the EIS very high measured resistance (membrane resistance)
c. The EIS current goes deeper in the living tissue interstitial fluid.
d. The electrode reaction is not an oxidation-reduction reaction, but is performed by chronoamperometry (Cottrell equation application) and therefore by physical diffusion of the chemical substance to the electrode surface.
e. EIS provided measurements
   The electronic box receives from the passive electrode the measurement of the intensity and voltage after passage into the interstitial fluid of the body and the digital analogic converter microchip transmits the data in numeric form (from 0-100) to the software which converts the data in resistance and conductivity in μSiemens.
f. Calculation in vivo of the interstitial fluid sodium ions density in 11 segments/pathways of the human body

The Cottrell Equation

$${c}_{o} = \frac{i}{n F A \sqrt{\frac{D}{π t}}}$$

i=measured intensity for each measured odd numbered segments
n=atomic number of Na+=11

### F=96500

A=electrode surface:

$$Forehead = 15.75 cm 2$$

$$Hand = 272 cm 2$$

$$Foot = 330 cm 2$$

$$D = \sqrt[3]{V atomic mass Na +} \text{=>} \sqrt[3]{V 22.98977} = 2.843$$

$$π = 3.14$$

$$t = time of tension = 1 second .$$

By the same way, we can calculate the interstitial fluid negative ions density.
g. The intensity and conductivity of the odd numbering segment is therefore proportional to the interstitial fluid Na+ ions density and according to the peer reviews about Na+/K+ATPase pump principle, the conductivity is proportional to the cellular mitochondrial ATP production.
h. The electrical Bioimpedance dispersion of the current (a parameter) is related with the morphology of the extra-cellular spaces.
i. The EIS estimation of the mitochondrial ATP production and the interstitial fluid morphology will be use in the hypoxia/ischemia detection.

### ES-BC Sensor to Estimate the Body Composition

### ES-BC Features

This technology is well known.

Following the sending of weak intensity at the mono frequency 50 KHz (to active tactile electrodes), the BIA sensor measures the resistance and reactance between 2 other passive tactile electrodes (tetra-polar mode).

The resistance and reactance calculate will be converting in estimated body composition parameters (TWB, Fat Free mass, fat mass) according to the predictive equations of BIA (Body Impedance Analysis) issue from the peer reviews.

### ESO Sensor Technology

### E.S.O Features

The E.S.O system is using the spectrophotometry technology (oximeter) with 3 features and signal processing analysis managed by software.

The Pulse Oximeter (SpO2 sensor) displays SpO2%, pulse rate value and vertical bar graph pulse amplitude.

The Photoelectrical Plethysmography or DPA (Digital Pulse Analysis) feature is the signal processing analysis of the pulse waveform provided by the oximeter.

The mathematical analyses provide indicators to estimate the hemodynamic parameters.

### The Heart Rate Detection Feature

Signal processing analysis of the heart rate variability: analysis both in the time domain and in the frequency domain (spectral analysis). Each QRS complex is detected and the so-called normal-to-normal (NN) or Rate-to-Rate (RR) intervals between adjacent QRS complexes are the result of sinus node depolarization.

The signal processing analysis of the measurement provides indicators to estimate the ANS (Autonomic Nervous System) activity.

### SpO2% Measurement:

### Pulse Oximeter

This technology is well known.
1) Fearnley, Dr S. J. “Pulse Oximetry.” Practical Procedures. Issue 5 (1995) Article 2: page 1. Available www.nda.ox.ac.uk/wfsa/html/u05/u05_{—}003.htm
2) “Introduction to the Pulse Oximeter.” www.monroecc.edu/depto/pstc/paraspel.htm

### Photoelectrical Plethysmography or DPA

This technology is well known. However this invention provides a new application in the calculation of the cardiac output measurement and hemodynamic indicators.

Estimated cardiac Output (Q) or (CO) FIG. 4

The cardiac output is calculated according to the

$$formula = \text{:} \frac{( 1 - \frac{{\sum }_{n = 2}^{N} {FFT}^{2} ( {f}_{n} )}{{\sum }_{n = 1}^{N} {FFT}^{2} ( {f}_{n} )} )}{( \frac{{S}_{2}}{{S}_{1}} )}$$

### Estimated SV=Stroke Volume

### SV=Q/HR

Where Q=cardiac output and HR=Heart rate

### Estimated BV=Blood Volume

### Calculation

Normal range according to the Nadler's Formula:

For Males=0.3669*Ht in M³+0.03219*Wt in kgs+0.6041
For Females=0.3561*Ht in M³+0.03308×Wt in kgs+0.1833

### Note:

*Ht in M=Height in Meters, which is then cubed
*Wt in kgs=Body weight in kilograms

And adjustment with the ECW (extracellular water) estimated from the E.S-Body composition device

### CI=Cardiac Index

### Cardiac Index (CI)=Q/Body Surface Area (BSA)

BSA (m²)=([Height (cm)×Weight (kg)]/3600)^{1/2}
Estimated EDV from the Blood Volume (BV)

### Estimated EF=Ejection Fraction

EF is proportional to the ejection time of the Second derivative PTG as follow:

EF (Ejection fraction) in %
From Ejection time (ET) SDPTG in ms

| ET | ET | EF |
|---|---|---|
| 400 | 500 | 35 |
| 350 | 400 | 40 |
| 320 | 350 | 42 |
| 310 | 320 | 45 |
| 305 | 310 | 52 |
| 290 | 305 | 55 |
| 280 | 290 | 58 |
| 260 | 280 | 60 |
| 250 | 260 | 65 |
| 240 | 250 | 68 |
| 200 | 240 | 70 |
| 190 | 200 | 72 |
| 180 | 190 | 75 |
| 100 | 180 | 80 |

MAP=Means Arterial Pressure from the Non Invasive blood pressure device
MAP=Diast. Pressure-((syst.-diast.)/3)

### Estimated SVR: Systemic Vascular Resistance

### SVR=(MAP/CO)×80

### Heart Rate Variability (HRV) Analysis

This technology is well known and provides indicator of the Autonomic nervous system activity level

### REFERENCE

Task Force of The European Society of Cardiology and The North American Heart rate variability Standards of measurement, physiological interpretation, and clinical use European Heart Journal (1996) 17, 354-381

### NIBP Sensor: Oscillometric Measurements

This type of device is in routine and does not need more clinical data and validation.

### Homeostasis Score.

### Homeostasis Score Using Bioimpedance, Spectrophotometry and Oscillometric Technologies: ES Teck Complex

1. Bioimpedance DC and Low Frequency Score Calculation
   The higher risk is the risk 1
   Graphic of the bioimpedance result and class risk
   For the body Abscissa=α parameter Ordinate=SDC in scale 0-100 corresponding to the conductivity values related to the body segments.
   Body risk: according to the zone number FIG. 5
   For the brain Abscissa=α parameter Ordinate: SDC in scale 0-100 corresponding to the conductivity value related to the brain segments.
   Brain risk: according to the zone number FIG. 6
   Calculation of the EIS Class Risk=0.75 Body risk+0.25 Brain risk

### If EIS HF>N=>Score −1

2. Photoelectrical Plethysmography or DPA Class Risk FIG. 7

### If SI>N or EF<N=>Score −1

3. HRV Class Risk. FIG. 8
   According to the zone number
4. SpO2% Class Risk
SpO2>=95 Class 5
SPo2>=99% Class 4
SPo2<95 and >=91: class 3
SPo2<91 and >80=>class 2
SpO2<80=>Class 1
5. Body Composition Class Risk
Normal range Class 5
FM>N+BMI<=29: Class 4
FM<N=>Class 3
FM>N+BMI>29 and <=35: Class 2
FM>N+BMI>35: Class 1
6. Blood Pressure Class risk

### Systolic <=120 Diastolic <=80=>Class 4

Systolic <=121-139 Diastolic <=81-89=>Class 3 pre-hypertension
Systolic <=140-159 Diastolic <=90-99=>Class 2 stage 1 hypertension
Systolic <=>160 Diastolic >100=>Class 1 stage 2 hypertension

### Homeostasis Score Calculation ES Teck Complex

### Maximum Score=30 FIG. 9

### Very Good=27-30

### Good=24-27

### Normal=20-24

### Warning=17-20

### Low=10-17

### Poor<10

### Homeostasis Score Spectrophotometry/ES-BC and Oscillometric Technologies: ESO

Same calculation for DPA, BC, HRV and NIBP
Maximum score=24

### Very Good=21-24

### Good=18-21

### Normal=0.15-18

### Warning=12-15

### Low=10-12

### Poor<10

The invention can further comprise additional or alternative monitoring devices to provde a medical device system as described herein.

It will be appreciated that the specific disclosures described and arbitrary scores assigned are illustrative to provide a working example and these can be altered significantly without departing from the essence of the invention as claimed.

1. A medical device system comprising at least two technologies wherein at least one technology is based on bio-impedance measuring and/or at least one technology is based on spectrophotometry measurements wherein software cross analyses the results to assess the homeostasis of an individual.

2. A system as claimed in claim 1 wherein a series of medical devices measure a variety of parameters using different technologies and software compiles the results of these to provide a homeostasis score.

3. A system as claimed in claim 1 comprising at least a pulse oximeter which provides a vascular waveform in combination with other biosensors and software.

4. A system as claimed in claim 1 which further includes EKG monitor, blood glucose meter, spirometer.

5. A system as claimed in claim 1 comprising at least 4 biosensors wherein signal processing-analysis is managed by software.

6. A system as claimed in claim 5 wherein the technologies include a) bioimpedance in bipolar mode (ElS sensor), b) bioimpedance in tetrapolar mode (ES-BC sensor), c) a spectrophotometer (ESO sensor) and d) oscillometric measurements. (NlBP sensor)

7. Use of a system as claimed in claim 1 to establish a homeostasis score for a patient.

8. A medical device system to assess the homeostasis of an individual, the system comprising bio impedance measuring equipment and spectophometry measuring equipment and software capable of analyzing both sets of results.

9. A medical device system as claimed in claim 8 wherein the bioimpedance measuring equipment measures bioimpedance in bipolar mode and in tetrapolar mode.

10. A medical device system as claimed in claim 9 wherein the spectophotmeter measuring equipment comprises a pulse oximeter.

11. A medical device system as claimed in claim 8 wherein the software analyzes the results to calculate assess a patient and provides the results as a homeostasis score.

12. A score of homeostasis of an individual comprising a series of defined tests including at least bioimpedance and spectrophotometer monitoring.
